(19) United States
(12) Patent Application Publication
(10) Pub. No.: US 2008/0014228 A1
(43) Pub. Date: Jan. 17, 2008

(54) Solid form

(76) Inventors: Olivia Darmuzey, Brussels (BE); Graeme MacLeod, Wezembeek Oppem (BE); Dzenana Cengic, Brussels (BE); Kevin M. Stokes, Hamilton Square, NJ (US)

Correspondence Address: PATENT ADMINISTRATOR;FMC CORPORATION, 1735 MARKET STREET, PHILADELPHIA, PA, 19103, US

(21) Appl. No.: 11/803,795

(22) Filed: May 16, 2007

Related U.S. Application Data
(60) Provisional application No. 60/830,912, filed on Jul. 14, 2006.

(51) Int. Cl. A61K 9/00; A61K 8/02; A61K 9/70; A61P 1/00; A61P 13/00; A61P 19/00; A61P 21/00; A61P 25/00; A61P 3/00; A61P 33/00; A61P 5/00; A61P 7/00; A61P 9/00

(52) U.S. Cl. 424/401; 424/439; 424/443; 426/282; 510/441

(57) ABSTRACT

An enrobed solid form comprising a film enrobing a compacted fill material having at least one active material, the solid form shows a weight loss that is less than 1% during a 30 minutes USP Friability Test, the fill material having a density of at least 0.5 g/ml based on the total solid volume of the solid form and a tensile strength less than 0.9 MPa, and the at least one active material within the solid form has an immediate release profile. The solid form is useful in effective delivery of high dose levels of active material.

### FIELD OF THE INVENTION

This application claims priority from U.S. Application No. 60/830,912 filed on Jul. 14, 2006, which is herein incorporated by reference in its entirety.

### BACKGROUND TO THE INVENTION

This invention relates to a solid form comprising a film enrobing a compacted fill material and a method of producing the solid form.

Active ingredients, for example pharmaceutical, agrochemical and detergent active ingredients may be delivered through a wide range of solid forms including tablets and capsules. Conventional tablets generally are highly compacted and have relatively high densities. In conventional tablets the active ingredient is generally compacted with other components to provide the requisite structural integrity for the tablet. Delivery of the active ingredient in use may however be unsatisfactory due to the effect of the compaction process and it is known to add excipients to the formulation to aid disintegration or dissolution of the tablet to improve delivery, aid compaction, increase strength and increase robustness of the solid form. This may however impose constraints on the flexibility of the formulator in developing tablets containing the active ingredient.

Capsules generally include the active ingredient in a relatively non-compacted form. However, the lack of compaction together with the void space inherent within capsules mean that for a given large dose of active, the volume of the final solid form is greater than for more compacted solid forms. Increasing the size of the capsule to accommodate the required dose is undesirable for the user. Typically, capsules require a relatively high level of disintegrant to provide adequate disintegration of the solid form. Capsule shells may also be sensitive to moisture and present problems as regards storage and product shelf-life.

WO 03/096963 discloses solid forms and processes utilizing films to enrobe a fill material to a degree of compaction less than that generally used to make a tablet. It is specifically disclosed therein that because of the nature of the capsule produced that certain ancillary ingredients necessary in conventional tablet production may be omitted. It is further disclosed therein that, due to relatively loose compaction, components contained within a tablet which are “designed to disperse and breakup the tablet when it has reached the site of delivery, can be omitted, as the active ingredients in the capsule according to the present invention are in a non-compacted or at least less compacted form as compared to a conventional tablet, and this lesser compaction leads to the easy release and dispersal of active ingredients once the capsule film has dissolved, e.g., at the intended site of delivery”.

There remains a need to provide a solid form able to provide a suitable dose level and to provide effective delivery of the active ingredient in use.

### SUMMARY OF THE INVENTION

The present inventors have found that a solid form having a compacted fill material with a particular combination of characteristics in which the compacted fill material is less compacted than in a tablet but more than in a capsule formulation provides beneficial delivery of the active ingredient at acceptable dose levels and with fewer or lower quantities of excipients typically employed in capsules or tablets.

The invention provides in a first aspect a solid form comprising at least one film enrobing a compacted fill material wherein:
i) said compacted fill material comprises at least one active material and at least one of a disintegrant and a wetting agent;
ii) said solid form shows a weight loss that is less than 1% during a 30 minutes United States Pharmacopeia (hereinafter referred to as USP) Friability Test USP 29 Test Number 1216 (page 3046); and
iii) said compacted fill material has a density of at least 0.5 g/ml based on the total solid volume of the solid form and a tensile strength of less than 0.9 MPa; and
iv) the at least one active material within the solid form exhibits immediate release.

### DETAILED DESCRIPTION OF THE INVENTION

Suitable active materials include a pharmaceutical active, food component or product, veterinary active, cosmetic component or product, an appetite suppressant, detergent component or product or nutraceutical component or product. Preferably, the solid form comprises at least one film enrobing a compacted fill material wherein the compacted fill material comprises at least one active material and at least one of a disintegrant and a wetting agent and the compacted fill material is selected from a pharmaceutical product, a food product, a veterinary product, a cosmetic, an appetite suppressant, a detergent product and a nutraceutical product, the said solid form shows a weight loss that is less than 1% during a 30 minutes United States Pharmacopeia (USP) Friability Test USP 29 Test Number 1216 (page 3046) and the said solid form dissolves with an immediate release profile.

The term “immediate release” as used herein refers to a solid form in which the active material is released from the solid form rapidly and wherein not less than 85% active material is released in 60 minutes, preferably in 45 minutes and especially in 30 minutes in the test specified in the USP Edition 29 Test Number 711 at page 2673 for said active material when said active material is placed in a dissolution medium as specified in the USP dissolution specification or selected from dissolution media specified in the USP according to the solubility properties of said active material. This is referred to in the USP as “Q” time. The term “immediate release” includes “fast release”.

The solid form comprises an active material which exhibits immediate release. The solid form may additionally comprise an active material which does not exhibit immediate release. If desired, the solid form may comprise an active material which exhibits immediate release and be free of an active material which does not exhibit immediate release.

In a preferred embodiment, the active material in the said solid form comprises at least one pharmaceutical active and said at least one pharmaceutical active has a mean dissolution which meets the USP dissolution specifications specified in USP Edition 29 Test Number 711 at page 2673 for said active material when said active material is placed in a dissolution medium as specified in the USP dissolution specification or selected from dissolution media specified in the USP according to the solubility properties of said active material.

Where a dissolution medium is specified in the USP for an active material, this is suitably employed in the dissolution test. Where there is either:
i) no USP test for the active material;
ii) more than one test for the active material; or
iii) the active does not meet the USP specification with the specified medium;
   the skilled person will select for the USP dissolution test the most appropriate medium from the USP dissolution media specified in the USP having regard to the dissolution characteristics of the active material.

Examples of media in which the dissolution test may be carried out include: (i) the medium specified in the USP preferably for said at least one active material, (ii) water, (iii) 0.1 M HCl or (iv) phosphate buffer having a pH between 5.8 and 8.0.

Preferably, the at least one of the active material has a mean dissolution of at least 75% in 300 seconds in the test specified in the USP Edition 29 Test Number 711 at page 2673 for said active material when the active material is placed in a dissolution medium as specified in the USP dissolution specification or selected from dissolution media specified in the USP according to the solubility properties of the active material or as selected by the skilled person for example selected from: (i) the USP for the at least one active material, (ii) water, (iii) 0.1 M HCI or (iv) phosphate buffer having a pH between 5.8 and 8.0.

A solid form having an active material meeting this dissolution test is considered herein to be a “fast release” solid form. The solid form preferably comprises an active material exhibiting a fast release. The solid form may comprise a further active material which does not exhibit fast release. As desired, the solid form does not contain an active material which does not exhibit a fast release.

Desirably, for immediate release solid forms and especially for high active levels, the active material has a rate of dissolution of at least 85% in 60 minutes or less, preferably 45 minutes or less, more preferably 30 minutes or less, and especially 15 minutes or less. Suitably, for fast release solid forms, at least 75%, preferably at least 80%, more preferably at least 85%, especially at least 90%, particularly at least 95%, more particularly at least 98% and most preferably at least 99% of the active material dissolves in 300 seconds, in the test specified in the USP Edition 29 Test Number 711 at page 2673 for said active material in a dissolution medium as specified in the USP dissolution specification or selected from dissolution media specified in the USP according to the solubility properties of the active material or as selected by the skilled person for example selected from at least one of: (i) the USP for the at least one active material, (ii) water, (iii) 0.1 M HCI or (iv) phosphate buffer having a pH between 5.8 and 8.0.

In an especially preferred embodiment the compacted fill material comprises at least one active material and at least one of a super disintegrant and a wetting agent and wherein the at least one of the active material has a mean dissolution of at least 75% in 300 seconds in the test specified in the USP Edition 29 Test Number 711 at page 2673 for said active material when the active material is placed in a dissolution medium as specified in the USP dissolution specification or selected from dissolution media specified in the USP according to the solubility properties of the active material or as selected by the skilled person for example selected from at least one of: (i) the USP for the at least one active material, (ii) water, (iii) 0.1 M HCI or (iv) phosphate buffer having a pH between 5.8 and 8.0.

The film enrobing the compacted fill material is preferably a water-soluble film.

Desirably, the film is in intimate contact with the compacted fill material. By “intimate contact” it is meant that the film and the compacted fill material are in direct contact preferably over the entire internal surface of film although some areas not being in direct contact with the compacted fill material may be acceptable. For example, a tablet or other product form may be contained within the film or the film may have a lining or coating presenting a barrier between the compacted fill and the film.

The compacted fill material is suitably compacted during the manufacture of the solid form. The compaction process is preferably carried out at lower compaction forces than conventionally applied in producing tablets.

The compacted fill material preferably has a density of less than 1.1 g/ml and more preferably less than 1.05 g/ml. The density of the compacted fill material is suitably at least 0.55 g/ml, Preferably, the density of the compacted fill material is from 0.55 to 1.04 g/ml, more preferably from 0.62 to 1.04 g/ml and desirably from 0.75 to 1 g/ml. The density of the solid form is suitably higher than that for conventional capsules and as the density contributes to the release profile of the solid form, this may be optimized by the formulator according to the release profile required.

The compacted fill material suitably has a tensile strength of less than 0.9 MPa, preferably less than 0.5, especially less than 0.2 MPa and particularly less than 0.1 MPa. The compacted fill has sufficient tensile strength to retain the physical integrity of the compacted fill and is preferably at least 0.05 MPa. The robustness of the solid form is suitably provided by the enrobing film rather than by the compacted fill.

In addition to having a density of at least 0.5 g/ml, preferably the said fill material has a porosity of less than 55%.

The compacted fill material also suitably has a porosity of at least 19%. Suitably, the porosity E may be calculated using equation (1):

(1) ε=1−(D_{A}/D_{T})×100 (%), where D_{A} is the density (g/ml) of the compacted fill material of the present invention based on the total solid form volume and D_{T} is the true density (g/ml) of the compacted fill material, which is a characteristic of each fill material and can be measured using a helium pycnometer. The total solid form volume comprises the internal volume defined by the enrobing film that is the compacted fill and any additional space within the film.

The solid form of the present invention has excellent robustness or physical strength. The robustness of a solid form may suitably be defined by measuring the weight loss of 10 solid forms when rotated in a USP friability apparatus. This test is as set out in USP 29 <1216> p 3046. The solid form of the present invention shows a weight loss of less than 1% when tested for 30 minutes in a friability drum. As conventional solid forms such as coated tablets are considered to be robust when the weight loss after 4 minutes of friability testing is less than 1% as set out in USP 29 <1216> p 3046, the solid form of the present invention is especially robust.

The density of the compacted fill material of the solid form of the present invention refers to the total weight of the compacted fill material divided by the total volume of the solid form within the film material. This is typically referred to as the “apparent” density of solid form. Unless otherwise stated or the context clearly requires, references to density herein are to “apparent” density.

The apparent density of a conventional tablet is typically greater than 1 g/ml as disclosed in, Pharmaceutical Technology, 27 (4), 67-80. In a conventional hard capsule, the fill material is lightly tamped so as to form a very weak slug that breaks up in the capsule shell, due to the air space above it. In a conventional hard capsule, the density of the fill material is therefore similar to the bulk density of the loose powder. The latter is typically less than 0.5 g/ml as disclosed in, Pharmaceutical Technology, 27 (4), 67-80. The density of the compacted fill material of the present invention is at least 0.5 g/ml and suitably at least 0.55 g/ml based on the total solid form volume.

A typical method for determining the density D of the compacted fill material in the present invention is to determine the fill weight W (2), the fill volume V, which depends on the size of the tooling used to manufacture the solid forms and to calculate D using equation (3):

W=Wt−Wf (g), where Wt is the weight of the total enrobed solid form and Wf is the weight of the film enrobing the solid form. (2)

D=W/V (g/ml) (3)

For a solid form of the present invention having a 70 microns thick film and made with oblong concave tooling of 16.6 mm length and 7.3 mm width, the volume V of the compacted fill material is calculated using equation (4):

V=(212.7+110.8t)/1000 (ml), where t is the sidewall thickness of the solid form (mm), typically measured using a micrometer. (4)

For a tablet or compact that is made using 13 mm diameter flat round punches, the volume V of the compacted fill material is calculated using equation (5):

V=[π(13/2)²t]/1000 (ml), where t is the tablet thickness (mm), typically measured using a micrometer. (5)

Conventional tablets are considered robust when the tensile strength of the compacted fill material is at least 1.0 MPa for example as disclosed in Pharmaceutical Technology, p52-62, April 2005 (Douglas McCormick,—Evolutions in Direct Compression). A typical method for determining the tensile strength for round flat faced cylinder shapes is to measure the crushing force (also called hardness) of compacts on a tablet hardness tester and calculate the tensile strength σ using equation (6) (Journal of Pharmaceutical sciences, vol. 59 (5), 688-691 Determination of table t strength by the diametral-compression test (Fell J. T. and Newton J. M., 1970)):

σ=2P/πDt (MPa), where P is the crushing force (N), D is the compact diameter (mm), and t is the compact thickness (mm), typically measured using a micrometer. (6)

The compacted fill material may be present at any desired level depending on the active material and the required dosage level. By way of example, the compacted fill material comprises active material at a level of 10 to 99.9% Suitably the solid form comprises a moderate to high amount of the active material and this will be selected according to the particular active material and the intended use of the product.

A moderate level of active material is suitably at least 30% and suitably up to 75%.

A high level if active material is suitably at least 75%, preferably at least 90%, and desirably not more than 99% for example 90 to 98%

The at least one active material may be in any form although in a preferred embodiment, the at least one active is a powder.

Suitably, the said solid form has a tensile strength of at least 1.3 MPa.

The compacted fill material may exhibit immediate release or fast release characteristics as desired. For fast release solid forms where the active material is not freely or very soluble as defined in the European Pharmacopaeia (EP), the compacted fill material preferably comprises at least one active material having a solubility of at least 1 g of active material in ≧10 g of water.

The present invention is also directed to a method of making the solid form of the present invention comprising providing a pocket-shaped thermoformed film having an open end, dosing through the open end a fill material into the pocket, compacting the fill material to provide a compacted fill material and enclosing the open end with a thermoformed film to provide the enrobed solid form.

Suitably the pocket-shaped film and the enclosing film are sealed to protect the compacted fill and desirably to provide a tamper-evident seal. The films are suitably sealed using an adhesive.

In a preferred embodiment, the inside surface of the enclosing film and the outside surface of the pocket-shaped film are sealed together.

The invention in a further aspect provides for the use of a solid form according to the invention in a method of treatment of the human or animal body by therapy. The invention also provides for the use of the solid form in the manufacture of a medicament for a method of treatment of the human or animal body by therapy.

Advantageously, solid forms according to the present invention in which the compacted fill material is enrobed in a film provide immediate release of the active material and preferably fast release.

The compacted fill material suitably comprises a disintegrant. Examples of suitable disintegrants include alginic acid, carboxymethylcelluloses, powdered cellulose, chitosan, colloidal silicon dioxide, guar gum, magnesium aluminium silicate, methylcellulose, microcrystalline cellulose, povidone, sodium alginate, starch, pregelatinised starch.

Super disintegrants are a type of disintegrant. In a fast release solid form the compacted fill material suitably comprises a super-disintegrant. The class of materials referred to as “super disintegrants” are known in the art and generally refer to such materials as crosslinked celluloses, crosslinked starches and crosslinked polymers. Examples of such include croscarmellose sodium, sodium starch glycolate, crospovidone, or low substituted hydroxypropyl cellulose.

The disintegrant may be used in an amount of 0.1 to 25% by weight of the compacted fill material, more particularly, 5 to 15% by weight, especially 8 to 12% by weight material, for example 10% by weight of the compacted fill material. The particular amount of disintegrant will be selected according to the particular disintegrant, formulation and use.

The super disintegrant may be used in an amount of 0.1 to 10% by weight of the compacted fill material, more particularly, 0.25 to 6% by weight, especially 1 to 4% by weight, for example 2 to 3% by weight of the compacted fill material.

Wetting agents may also be used in the compacted fill material of the present invention. The class of materials referred to as “wetting agents” are well known in the art and generally refer to such materials that are usually surface-active materials or surfactants, which reduce the contact angle between solid and liquid and therefore increase the adhesion of the liquid to the solid surface of an active material. Examples of such include hypromellose, docusate sodium, sodium lauryl sulfate, polyoxyethylene sorbitan fatty acid esters, sorbitan esters, polyoxyethylene alkyl ethers, dioctyl calcium sulfosuccinate. Other examples of wetting agents include solubilizing agents such as povidone, cyclodextrins, poloxamers, glyceryl monostearate.

The wetting agent is suitably used in an amount of 0.01 to 10% by weight of the compacted fill material, more particularly, 0.1 to 2% by weight.

Unless otherwise stated, where reference is made herein to a dissolution test or characteristics, these are determined according to USP dissolution test specified in USP Edition 29 Test Number 711 at page 2673. Where there is either:
i) no USP test for the active material;
ii) more than one test for the active material; or
iii) the active does not meet the USP specification with the specified medium;
   the skilled person will select the most appropriate medium for the dissolution test from the USP dissolution media specified in the USP having regard to the dissolution characteristics of the active material and carry out a dissolution test according to the procedure of USP Test Number 711.

A wide range of active materials having widely differing solubility characteristics may be employed in the present invention. The active material may have a solubility in water of 1 g in less than 1 g water, 1 g in 1 to 10 g water, 1 g active in 10 to 30 g water, 1 g active in 30 to 100 g water, 1 g active in 100 to 1,000 g water, 1 g active in 1,000 to 10,000 g water, and 1 g active in more than 10,000 g water.

Any solid active material having the above water solubilities may be used in the present invention alone or in combination provided that it meets the USP dissolution test specified in USP Edition 29 Test Number 711 at page 2673.

Examples of suitable classes of pharmaceutical actives include an analgesic, antiangina, antianaemia, antibiotic, antiarrhythmic, antidiarrheal, antidiuretic, antidepressant, antiemetic, antifungal, antirheumatic, antiviral, antiprotozoal, antihistamine, antihypertensive, anti-inflammatory, antimigraine, antinausea, antispasmodic, anxiolytic, beta blocker, calcium channel blocker, sedative, hypnotic, antipsychotic, bronchodilator, decongestant, cough expectorant, cough suppressant, antiasthma drug, corticosteroid, actives for treatment of cough or common cold, muscle relaxant, erectile dysfunction active, motion sickness active, anti-HIV, anti-malaria actives, anti-cholesterol actives, respiratory actives, gastronintestinal actives, cardiovascular actives, antidiabetes actives, central nervous system actives, anti-infection actives, mucolytics, proton pump inhibitor and nasal decongestants.

Examples of suitable actives include paracetamol, pseudoephedrine, acravastine, lamivudine, abacavir, pravastatin, Roziglitazone, ezetimibe, Clavulanate, sulfamethoxazole, benazepril, Valsartan, Irbesartan, Losartan, Dutasteride, tamsolusin, Atazanavir, ritonavir, propoxyphene, Hydrocodone, Metocarbamol, Memantine, Donepezil, Glyburide, Pioglytazone, Glimepiride, Benazepril, Torcetrapib, Eprosartan, Telmisartan, Olmesartan, Lopinavir, Emtricitabine, Tenofovir, Amprenavir, Tipranavir, Atovaquone, Proguanil, 5-ASA acid, 4-APA acid, Bismuth citrate, Bismuth subsalicylate, Montelukast, pseudoephedrine, Guaifenesin, ibuprofen, nifedipine, betamethasone acetate, methylprednisolone, dextromethorphan, cinnarazine, simvastatin, ciprofloxacin, glipizide, risperidone, glibenclamide, fenofibrate, isosorbide mononitrate, isosorbide dinitrate, acetazolamide, levothyroxine sodium, omeprazole, aspirin, codeine, dihydroergotamine, diazepam, theophylline, sildenafil citrate, vardenafil hydrochloride, amlodipine besylate, zolpidem tartrate, acetaminophen, methocarbamol, ramipril, digoxin, enalapril maleate, fluoxetine hydrochloride, fexofenadine hydrochloride, olanzapine, methyldopa, hydrochlorothiazide, timolol maleate, alendronate sodium, thiabendazole, rofexocib, dicoflenac, bepridil hydrochloride, atorvastatin hydrochloride, sertraline hydrochloride, famciclovir monohydrate, nabumetone, cimetidine, ketoprofen, etodolac, amiodarone hydrochloride, indomethacin, cefaclor, diltiazem, verapamil, felodipine, isradipine, nicardipine, prazosin, disopyramide, pentoxifilline, venlafaxine, alfuzosin, doxazosin, famotidine, ranitidine, pirenzipine, lansoprazole, loperamide, sulfasalazine, prednisolone, furosemide, amiloride, triamterene, verapamil, atenolol, propranolol, captopril, glyceryl trinitrate, caffeine, aminophylline, cetirizine, loratadine, chlorpheniramine maleate, diphenhydramine, dothiepin, amitriptyline, pheneizine, paroxetine, fenfluramine, dimenhydrinate, ondansetron, domperidone, metoclopramide, tramadol, dihydrocodeine, pethidine, sumatriptan, amoxicillin, ampicillin, cefuroxime, cephalexin, tetracycline, erythromycin, co-trimoxazole, sulphadiazine, trimethoprim, nitrofurantoin, fluconazole, ketoconazole, acyclovir, zidovudine, chloroquine, mefloquin, metronidazole, metformin, chlorpropamide, ferrous sulphate, azapropazone, fenbufen, flurbiprofen, ketoprofen, naproxen, piroxicam, mefanamic acid, celecoxib, licofelone, tadalafil, mycophenolate, valgancyclovir, valacyclovir, sevelamer, metaxolone, nelfinavir, duranavir, tipranavir, levetiracetam, capecitabine, moxifloxacin, morphine, levofloxacin, clarithromycin, pregabalin, esomeprazole, quetiapine, efavirenz, oxcarbazepine, colesevelam, zileuton, nitazoxanide, clofibrate, praziquantel, sucralfate, cefprozil, indinavir, ganciclovir, oxaprozin, divalproex, cefadroxil, felbamate, potassium chloride, saquinavir, fosamprenavir, hydroxyurea, gabapentin, niacin, omega-3 acid ethyl esters, calcium acetate, progesterone, procainamide, delavirdine, ribavirin, propafenone, eprosartan, tocainide, tinidazole, choline magnesium trisalicylate, azithromycin, linezolid, lorazepam, oxazepam, lormetazepam, flunitrazepam, haloperidol, triptorelin, leuprorelin, lanreotide acetate, octreotide acetate, methylxanthin, tamsulosin, codeine hydrochloride, dextromoramide tartrate, ethymorphine hydrochloride, magnesium salicylate, methadone hydrochloride, oxycodone hydrochloride, sufentanil citrate, ephedrine, tramazoline hydrochloride, brompheniramine maleate, emedastine fumarate, and pharmaceuticaly or nutraceuticaly acceptable salts, acids, esters, isomers, and metabolites thereof.

Where more than one active material is present, the two or more actives may be from the same class or may be from different classes. Examples of combinations of active materials from different classes include an antibiotic in combination with one of a decongestant, an anti-inflammatory, a cough expectorant, a cough suppressant or an active for treatment of cough or common cold, a proton pump inhibitor, anti-hypertension and anti-cholesterol actives.

Examples of classes where two or more active materials from one class may suitably be employed include respiratory actives, gastronintestinal actives, cardiovascular actives, antidiabetes actives, central nervous system actives, anti-infection actives, anti-viral actives, analgesics, anti-inflammatory actives, antibiotics, cough suppressants, expectorants, mucolytics, and nasal decongestants, anti-HIV, anti-malaria actives.

Examples of particular combinations of active materials include: Paracetamol and Caffeine; Aspirin and paracetamol; Paracetamol and pseudoephedrine; Paracetamol and phenylephrine; Ibuprofen and codeine; Ibuprofen and pseudoephedrine; Paracetamol and diphenhydramine; Acravistine and pseudoephedrine; Paracetamol and dextromethorphan; Parcetamol and guaphenesin; Paracetamol, caffeine, aspirin; Aspirin and caffeine; Zidovudine, lamivudine and abacavir; Pravastatin and aspirin; Lamivudine and zidovudine; Roziglitazone and Metformin; Ezetimibe and fenofibrate; Amoxicillin and Clavulanate; Trimetoprim and sulfamethoxazole; Amlodipine and benazepril; Valsartan and Hydrochlorothiazide; Irbesartan and Hydrochlorothiazide; Losartan and Hydrochlorothiazide; Fenofibrate and Metformin; Abacavir and lamivudine; Dutasteride and tamsolusin; Atazanavir and ritonavir; Ritonavir and Saquinavir; Propoxyphene and paracetamol; Hydrocodone and paracetamol; tramadol and paracetamol; Metocarbamol and paracetamol; Memantine and Donepezil; Glyburide and Metformin; Pioglytazone and Metformin; Rosiglitazone and Glimepiride, Benazepril and Hydrochlorothiazide; Atorvastatin and Torcetrapib; Eprosartan and Hydrochlorothiazide; Amlodipine and Atorvastatin; Ezetimibe and Simvastatin; Telmisartan and Hydrochlorothiazide; Olmesartan and Hydrochlorothiazide; Lopinavir and Ritonavir; Emtricitabine and Tenofovir; Fosamprenavir and Ritonavir; Amprenavir and Ritonavir; Tipranavir and Ritonavir; Atovaquone and Proguanil; Lansoprazole, Amoxicillin and Clarithromycin; Lansoprazole and Naproxen; 5-ASA and 4-APA acid; Clarithromycin, Ranitidine and Bismuth citrate; Bismuth subsalicylate, Metronidazole and Tetracycline; Montelukast and Loratadine; Fexofenadine and pseudoephedrine; Guaifenesin and pseudoephedrine.

Low levels of active, for example from 1 to 30% may be employed as desired. However, the amount of the active material present in the compacted fill material is suitably at least 30% by weight of the compacted fill material, preferably at least 40%, more preferably at least 50%, particularly at least 60%, more particularly at least 70% and especially at least 75% by weight of the compacted fill material. In embodiments in which a high dose of active material is required, the amount of active material is desirably at least 80% by weight of the compacted fill material, more desirably at least 85%, particularly at least 90%, more particularly at least 95%, and especially at least 99%.

The active material of the present invention is preferably a powder and this suitably includes such powders as granules, micronized powders, spray-dried powders, freeze-dried powders and pellets.

The compacted fill material of the present invention may contain at least one filler. Examples of the filler include a broad category of excipients such as glidants, binders and lubricants. Examples include microcrystalline cellulose, dicalcium phosphate, lactose, calcium carbonate, calcium phosphate dibasic anhydrous, calcium phosphate dibasic dehydrate, calcium phosphate tribasic, powdered cellulose, silicified microcrystalline cellulose, cellulose acetate, compressible sugar, confectioners sugar, dextrin, dextrose, ethylcellulose, fructose, lactitol, starch, pregelatinized starch, sucrose, talc, xylitol, maltodextrin, magnesium carbonate, maltose, mannitol, polydextrose, sodium alginate, sodium chloride, sorbitol, sucrose, sugar spheres, acacia, carrageenan, carbomer, chitosan, hydroxypropylmethylcellulose, carboxymethylcellulose sodium, gelatin, guar gum, hydroxyethyl cellulose, hydroxypropyl cellulose, methylcellulose, povidone, zein, citric acid, sodium bicarbonate, alginic acid, carboxymethylcellulose calcium, colloidal silicon dioxide, low substituted hydroxypropyl cellulose.

When a filler is present, it may be present in an amount of less than 70% by weight of the compacted fill material, less than 60% by weight, suitably less than 50% by weight, may be less than 40%, may be less than 30% by weight, for example from 1 to 25% by weight, from 1 to 10% by weight and from 1 to 5% by weight of the compacted fill material. As desired, the enrobed solid form of the present invention may contain no filler (except where the filler is a disintegrant) in the compacted fill material.

The film to be used to enrobe the present invention may be any film capable of enrobing the compacted fill materials without adversely impacting the desired dissolution profile. The film to be used may comprise water soluble components, water insoluble components or may comprise soluble and insoluble components in combination.

Preferably, the compacted fill material of the present invention is enrobed by a film comprising at least one water soluble polymer. Films generally useful in the present invention include those that are thermo formable and generally have dissolution rates appropriate for the preparation of rapid release, preferably immediate release, solid forms of the invention. Examples of such water soluble polymers include cellulosic materials such as hydroxyethyl cellulose, hydroxypropyl cellulose, hydroxypropyl methyl cellulose; polyvinyl alcohol; hydrocolloids such as carrageenan, alginate and pectin; and water soluble acrylates. Examples of water insoluble polymers include ethylcellulose, methacrylates and cellulose acetate. The films used in the invention may be gelatin free. The films may contain plasticizers such as lactic acid, citric acid, polyethylene glycol, sorbitol, glycerine, triethylcitrate, propylene glycol, phthalates, triglycerides, triacetin, tributylcitrate, etc. WO 2004/026284, WO 02/083779 and WO 03/095548 disclose further examples of films that may be used in the invention and such are incorporated herein by reference. Examples of films that may be used in the present invention are available under the trade name XGEL UNO from BioTec Films LLC, Tampa, Fla., US. Films for use in the present invention may be made in a conventional manner. If desired, an adhesive and use thereof can be used to aid in sealing the films together

If desired, an adhesive and use thereof can be used to aid in sealing the films together. Suitable adhesive compositions include those set forth in WO 04/10337 and WO 04/103338—both of which are incorporated herein by reference.

The solid forms of the present invention may be enrobed and prepared in accordance with the methods disclosed in WO 03/096963, WO 05/030115, WO 05/030116 and PCT/GB2005/001077—all of which are incorporated herein by reference.

### BRIEF DESCRIPTION OF THE DRAWINGS

The present invention will now be further described with reference to specific examples which are illustrative of the invention.

FIG. 1 represents the ibuprofen dissolution profile of various samples from Example 1.

FIG. 2 represents the ibuprofen dissolution profile of various samples from Example 5.

FIG. 3 represents the acetaminophen dissolution profile of various samples from Example 6.

In this specification, all parts and percentages are by weight unless otherwise noted.

### EXAMPLES

MATERIALS

| Material | Grade | Supplier |
|---|---|---|
| acetaminophen DC-90 | COMPAP ® L | Mallinckrodt Pharmaceuticals |
| (90% acetaminophen) | | Greensboro NC |
| acetaminophen (APAP) | Rhodapap ® paracetamol | Rhodia Pharma Solutions |
| | powder | Cranbury NJ |
| Aspirin | Crystals USP grade | JiLin HengHe Pharmaceutical |
| | | Co., Ltd. |
| Ketoconazole | EP grade | Bufa B.V. |
| Theophylline | USP grade | Shandong Xinhua |
| | | Pharmaceutical Co., Ltd. |
| benzyl alcohol | | EM Science |
| croscarmellose sodium | AC-DI-SOL ® | FMC Corp, Philadelphia PA |
| Docusate Sodium | USP grade | Sigma |
| Sodium Lauryl Sulfate | | Merck |
| dicalcium phosphate | Di-Tab ® | Rhodia Pharma Solutions |
| dihydrate (DCP) | | Cranbury NJ |
| hypromellose (HPMC) | Methocel ® E 15LV | Dow Chemical |
| Ibuprofen | BP/EP grade, mean | Shasun Chemicals & Drugs |
| | particle size: 67.5 microns | Ltd |
| Ibuprofen | 115 | Ablemarle |
| magnesium stearate | | Brenntag |
| microcrystalline cellulose | Avicel ® PH 200 | FMC Corp, Philadelphia PA |
| microcrystalline cellulose | Avicel ® PH 102 | FMC Corp, Philadelphia PA |
| microcrystalline cellulose | Avicel ® PH 101 | FMC Corp, Philadelphia PA |
| Lactose | Lactopress ® anhydrous | Borculo Domo Ingredients |
| | | Zwolle Netherlands |
| silica (colloidal) | Cab-o-Sil ® M-5P | Cabot Corp, Boston MA |
| Starch | Hi Set ® C | National Starch and |
| (55% amylose, modified) | | Chemical, Bridgewater NJ |
| Talc | | Luzenac, Paris France |
| Talc | EP/BP grade | VWR |
| Triacetin | | Eastman |

### Methods

Fill material: The ibuprofen and theohylline active powders were sieved through a 24 mesh screen (710 microns) prior to weighing. The Rhodapap® acetaminophen active powder was sieved through a 35 mesh screen (425 microns) prior to weighing. Powders were weighed out and blended for 15 to 20 minutes in a Turbula TF2 shaker mixer, a PharmaTech V-blender or in a Speedmixer DAC150FVZ-K for 5 seconds at 3000 rpm. The powder fill material was stored in a plastic bottle or double plastic bags until use. Ibuprofen granulate was prepared using a 4M8 granulator from Pro-Cept: Ibuprofen and Avicel were weighed out and blended in the granulator for 5 minutes at 500 rpm, 145 ml of a solution of 8.8 g/ml Docusate Sodium in DI water was added at a speed of 11 ml/min in the 297 mg powder blend, which was mixed at 1000 rpm. The granules were then dried in an oven at 50° C. for 12 hours and screened through a #12 sieve thereafter. Ibuprofen and ketoconazole are practically insoluble in water (1 g in more than 10,000 g of water), acetaminophen is sparingly soluble in water (1 g in 30 to 100 g of water), aspirin and theophylline are slightly soluble in water (1 g in 100 to 1000 g water).

Powder compacts: Powder compacts of varying densities were prepared by compressing the powder fill material in an ESH compaction simulator (ESH Testing Ltd, Dudley, UK) with flat round punches having a diameter of 13 millimeters. The upper punch speed was 100 mm/sec giving a 0.36 second cycle. The upper punch displacement was adjusted to vary the compaction force. The maximum applied force was chosen to be lower than a force that would result in mechanical failure (“capping”) of the compressed fill formulation. Six compacts were prepared for each test condition. The amount of powder fill used was 250 milligrams for compaction pressures of 30 MPa or less and 500 milligrams for compaction pressures of more than 30 MPa. The upper punch force and compact weight were recorded. The compaction pressure reported was based on the upper punch force divided by the punch area (132.73 square millimeters). Compacts were stored in double plastic bags. The compact thickness measured prior to the beginning of the dissolution testing was used to calculate the apparent density.

Enrobed solid form: A soluble film known as XGEL UNO and supplied by Bio Tec Films LLC was cut into strips 6 centimeters by 20 centimeters. The lower film had a thickness of about 120 microns and the upper film had a thickness of about 80 microns or 120 microns. The lower film was heated sufficiently to thermoform under vacuum into dose cups about 2 to 4 millimeters in height to conform to cavities (7.5 millimeters width by 16.75 length millimeters) with the cavity depth determined by height-adjustable dose-shaped lower pistons within the stainless steel die. The film strip was placed over the die and brought in contact with a heated TEFLON® coated surface by means of upward vacuum. The film was then drawn into the stainless steel die cavities by inverting the vacuum to form a strip of twelve thermoformed dose cups with 3.0 millimeters separation between adjacent dose cups. Some unused portion of the filmstrip was cut and removed. The fill composition was dosed (by volume) into the dose cups, then the fill was lightly compacted in the dose cups with upper pistons, and the lower film was cut to separate the individual solid forms. The solid forms were then lifted by the lower pistons to expose a portion of the solid form sidewalls for application of the upper film to complete the enrobing of the solid form. An adhesive composed of 5% Methocel E15LV Premium, 45% Benzyl alcohol and 50% Triacetin, [was applied (by transfer roller) to the upper filmstrip on the side to be pressed against the exterior sidewall of the dose cup. This adhesive and application method is referred to as “Adhesive 1” in the examples. Alternatively, an adhesive composed of 62% Benzyl alcohol, 31% Ethanol, 5% Potassium Acetate, 2% Deionised Water and Erythrosine was sprayed on the upper portion of the exterior sidewall of the lower film prior to the upper thermoforming step. This adhesive and application method is referred to as “Adhesive 2” in the examples. The upper film was placed over the solid forms containing the compressed powder fill and the film was heated by contact with the heating element using upper vacuum. The heated upper film was formed around the solid forms using the lower vacuum enclosing the fill material within the solid form by overlapping the upper film onto the sidewall of the solid forms. The top film was cut to separate the completed enrobed solid forms and the unused film was removed. Each solid form was ironed by forcing it through a heated die under low pressure so that the cut film overlapping the sides was pressed smooth. Examples 1, 5, 6, 7-1, 8-1, 8-2, 11-2, 11-3 and 11-4 below used the apparatus set forth in WO 2005/030115, and examples 7-2, 7-3, 7-4, 9-1, 9-2, 9-3, and 11-1 below used the apparatus set forth in WO 2005/030116.

Dissolution tests were carried out according to USP 24 and USP 28 with dissolution apparatus 2, paddles. Disintegration tests were carried out according to USP 28, with disks.

### Example 1

A powder fill was prepared with the following composition: 74.5% Ibuprofen, 20% AVICEL® PH 200, 4.0% AC-DI-SOL®, 1.0% talc and 0.5% magnesium stearate. The ibuprofen, Avicel and Ac-Di-Sol were mixed together for 20 minutes in a V-blender. The talc and magnesium stearate were then added and mixed for 10 minutes. This powder fill material was used to fill two types of commercial hard shell capsules: gelatin hard shell capsules (Capsugel size 0) and hydroxypropyl methylcellulose hard shell capsules (Shionogi size 0); and to prepare an enrobed solid form of the present invention. The capsules were filled by hand with a semi-automatic capsule filling machine. Both the upper and lower films were 120 microns in thickness. The thermoforming steps were at 140° C. for 2 seconds. Adhesive 1 was used. The ironing step was 30 seconds at 45° C. Table 1 shows the weights of the solid form and its components (the shell or film, and the fill material), the mean ibuprofen content of the powder fill material, the disintegration time, and the percentage of ibuprofen released at 5 minutes in the dissolution test at 37° C. according to USP 24 for Ibuprofen immediate release tablets using 900 ml of phosphate buffer at pH 7.2 in dissolution apparatus 2, paddles.

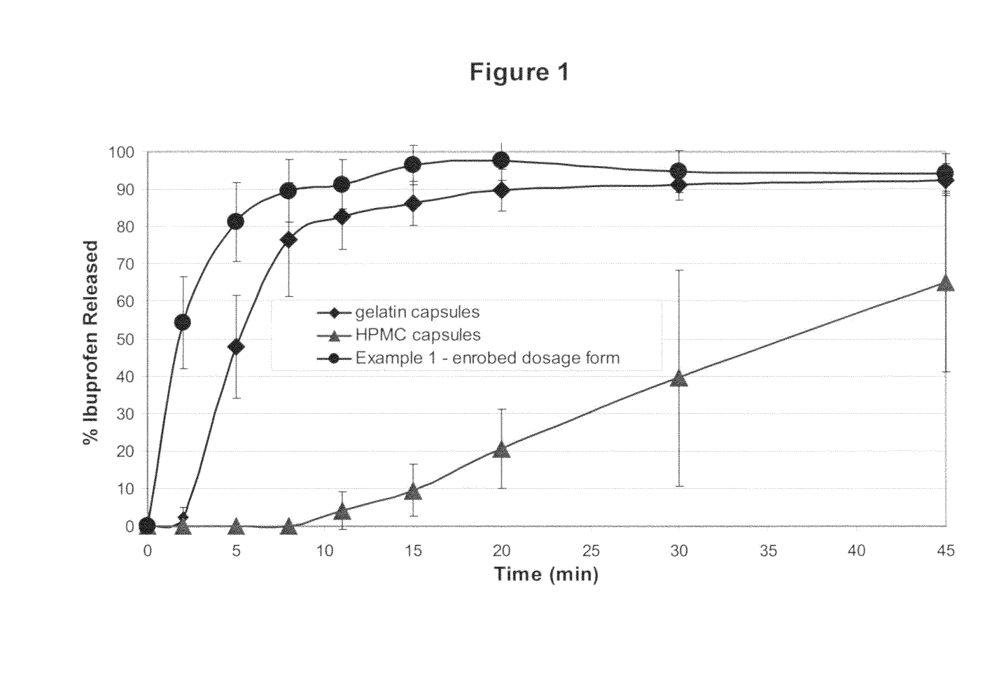

The release of the ibuprofen using the same powder fill material was significantly faster from the enrobed solid form of the present invention than from the commercial gelatin or HPMC hard capsules as shown in Table 1 and FIG. 1.

TABLE I
Ibuprofen release at 5 minutes from solid forms
with a 74.5% ibuprofen powder fill

| | Example 1 | Gelatin | HPMC |
|---|---|---|---|
| | enrobed dose | hard shell | hard shell |
| | present invention | capsule | capsule |
| Solid form weight | 541.8 | 427.2 | 431.4 |
| (mg) | | | |
| Shell weight (mg) | 44.9 | 94.0 | 89.7 |
| fill weight, net (mg) | 496.9 | 333.2 | 341.7 |
| Ibuprofen (mg) | 370 | 248 | 255 |
| Disintegration time | 25 sec | 2 min 14 sec | 3 min 30 sec |
| Dissolution test | Ibuprofen (%) | Ibuprofen (%) | Ibuprofen (%) |
| 300 seconds | 81 +/− 11 | 48 +/− 14 | 2 +/− 1 |

### Example 2

Dissolution of compacted and non-compacted powder fill compositions containing 76% ibuprofen with and without AC-DI-SOL® croscarmellose sodium was measured in phosphate buffer at pH 7.2 as specified in USP 24 for ibuprofen. USP specifications for Ibuprofen tablets for immediate release are: not less than 85% of the drug dissolved after 60 minutes (Q). This is referred to as the “Q-time.”

The powder fill compositions were: (1) 76% ibuprofen, 23% AVICEL® PH200 microcrystalline cellulose, and 1% talc and (2) 76% ibuprofen, 20% AVICEL® PH200, 3% AC-DI-SOL® and 1% talc. The powder compacts were prepared using 250 mg of powder fill when the compaction pressure was 30 MPa or less and 500 mg when the compaction pressure was greater than 30 MPa. The non-compacted powders were tested for dissolution using 500 mg of the powder fill composition.

Table II shows the percentage of ibuprofen released during dissolution at 5 minutes as a function of density for samples with and without the AC-DI-SOL® croscarmellose sodium. The samples containing 3% Ac-Di-Sol in Table II are all examples within the scope of the invention.

TABLE II
Effect of fill density on ibuprofen release

| Compaction | | Ibuprofen (%) | | Ibuprofen (%) | |
|---|---|---|---|---|---|
| Pressure | Density | 0% Ac-Di-Sol | | 3% Ac-Di-Sol | |
| (MPa) | (g/ml) | 5 min | Q (60 min) | 5 min | Q (60 min) |
| 68 | 1.05 | 5 ± 1 | 26 ± 4 | 73 ± 6 | 90 ± 1 |
| 30 | 0.98 | 11 ± 2 | 36 ± 7 | 89 ± 5 | N/A |
| 20 | 0.92 | 8 ± 5 | 53 ± 6 | 88 ± 9 | N/A |
| 10 | 0.84 | 60 ± 21 | 83 ± 2 | 80 ± 14 | 86 ± 1 |
| 0 | 0.48 | 50 ± 8 | 85 ± 1 | 61 ± 7 | 85 ± 1 |

### Example 3

Powder compacts containing 76% ibuprofen were prepared using fill formulations as in Example 2 except replacing the microcrystalline cellulose (a viscoelastic, insoluble filler) with lactose (a brittle, soluble filler) or dicalcium phosphate dihydrate (a brittle, insoluble filler). The dissolution data for the microcrystalline cellulose are from Table II in Example 2. All Ibuprofen compacts were tested for dissolution at 37° C. according to USP 24 for Ibuprofen immediate release tablets using 900 ml of phosphate buffer at pH 7.2 in dissolution apparatus 2, paddles. USP specifications for Ibuprofen tablets for immediate release are: not less than 85% of the drug dissolved after 60 minutes (Q). The samples containing 3% Ac-Di-Sol in Table III are all examples within the scope of the invention.

TABLE III
Effect of Filler Type on Drug Release

| | | | | | Ibuprofen |
|---|---|---|---|---|---|
| | | | Ibuprofen (%) | | (%) |
| | Compaction | Density | 0% Ac-Di-Sol | | 3% Ac-Di- |
| Filler | Pressure (MPa) | (g/ml) | 5 min | Q (60 min) | Sol at 5 min |
| MCC | 20 | 0.92 | 8 ± 5 | 53 ± 6 | 88 ± 9 |
| Lactose | 5 | 0.88 | 9 ± 1 | 77 ± 16 | 86 ± 15 |
| DCP | 5 | 0.92 | 3 ± 7 | 21 ± 15 | 79 ± 4 |

### Example 4

Powder compacts containing 76% ibuprofen were prepared using the fill material formulations of Example 2 but with varying amounts of AC-DI-SOL® croscarmellose sodium. As the amount of AC-DI-SOL® was decreased in the formulation, it was replaced with additional AVICEL® PH 200 microcrystalline cellulose. Compacts containing all levels of AC-DI-SOL® tested had an increased amount of ibuprofen released at 5 minutes of dissolution when tested for dissolution at 37° C. according to USP 24 for Ibuprofen immediate release tablets using 900 ml of phosphate buffer at pH 7.2 in dissolution apparatus 2, paddles compared to the powder compact without AC-DI-SOL® as shown in Table IV. All samples in Table IV are examples within the scope of the present invention.

TABLE IV
Effect of Ac-Di-Sol Level on Drug Release at 5 minutes

| Ac-Di-Sol level (%) | Density (g/ml) | Ibuprofen (%) |
|---|---|---|
| 0.25 | 0.77 | 86 ± 6 |
| 0.5 | 0.76 | 89 ± 7 |
| 1.0 | 0.77 | 90 ± 9 |
| 2.0 | 0.77 | 88 ± 9 |
| 3.0 | 0.78 | 99 ± 3 |

### Example 5

Commercial solid forms containing 200 milligrams of ibuprofen were tested for dissolution. The commercial products tested were NUROFEN® sugar coated tablets, NUROFEN® liquid filled gelatin softgel capsules, and ADVIL® (bicolored) gelatin dipped caplets.

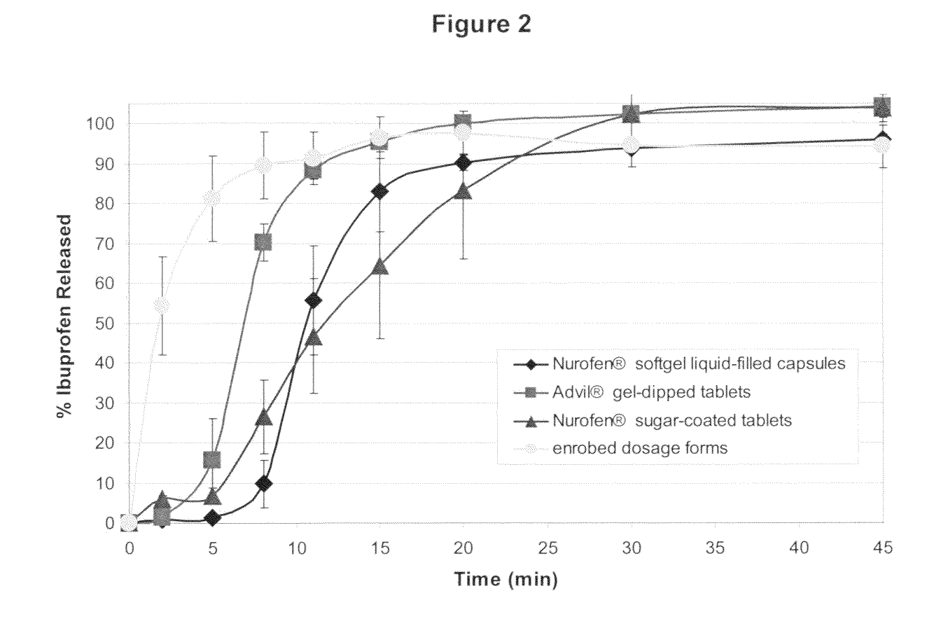

Table V shows that the ibuprofen release was significantly faster from the enrobed solid form (Example 1) of the present invention than from the tested commercial solid forms. FIG. 2 shows that the time required for approximately 80% ibuprofen release was about 5 minutes for an enrobed dosage (Example 1) of the present invention, about 10 minutes for the ADVIL® gelatin dipped tablets, about 15 min for the Nurofen® liquid filled softgel capsules, and about 20 minutes for the Nurofen® sugar coated tablets.

The Ibuprofen solid forms were tested for dissolution at 37° C. according to USP 24 for Ibuprofen immediate release tablets using 900 ml of phosphate buffer at pH 7.2 in dissolution apparatus 2, paddles.

TABLE V
Ibuprofen release at 5 minutes from enrobed
dose vs commercial solid forms

| | Solid form | | | |
|---|---|---|---|---|
| | Enrobed | | | |
| | Solid form | | | |
| | Present | NUROFEN ® | NUROFEN ® | ADVIL ® |
| | Invention | Sugar coated | softgel | gel dipped |
| | (Example 1) | tablets | capsules | tablets |
| Ibuprofen | 81 +/− 11 | 7 ± 2 | 2 ± 1 | 16 ± 10 |
| (%) | | | | |
| released in | | | | |
| 5 minutes | | | | |

### Example 6

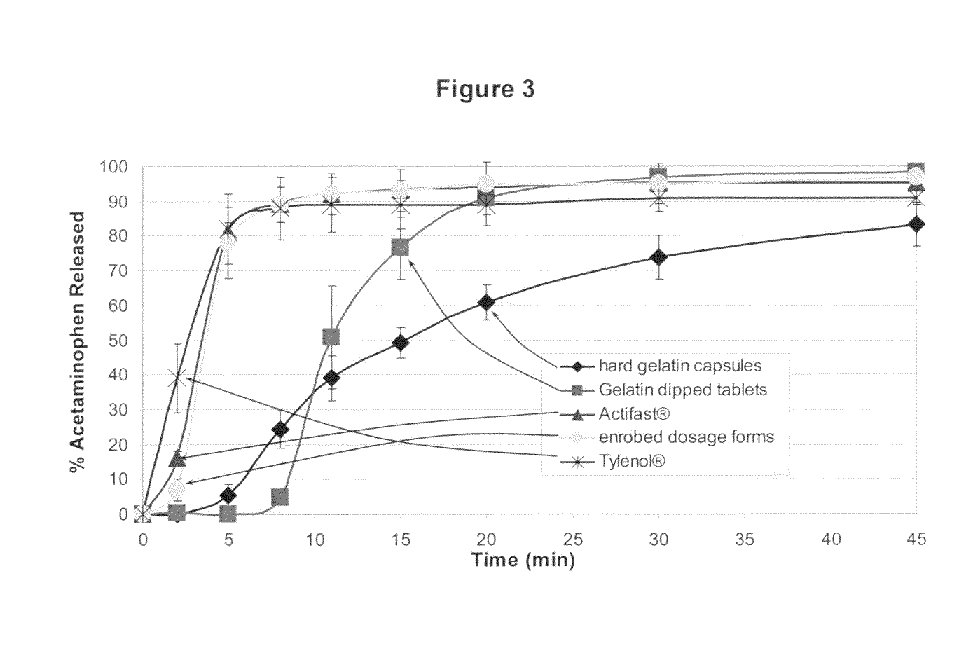

Commercial solid forms containing 500 mg dose of acetaminophen were tested for dissolution at 37° C. according to USP 24 for acetaminophen using 900 ml of phosphate buffer at pH 5.8 in dissolution apparatus 2, paddles. The commercial solid forms were hard shell gelatin capsules manufactured by Boots, TYLENOL RAPID RELEASE® gelcaps, PANADOL ACTIFAST® film coated tablets, and bicolored gelatin-coated capsule-shaped tablets (“geltabs”) manufactured by Banner Pharmacaps. Enrobed solid forms of the present invention containing acetaminophen were prepared as in Example 1 using 441 mg of COMPAP® L as the powder fill material. FIG. 3 shows the acetaminophen dissolution profile after 2 minutes, 5 minutes, 8 minutes, 11 minutes, 15 minutes, 20 minutes, 30 minutes, and 45 minutes. Data for % acetaminophen release at 5 minutes dissolution are in Table VI.

TABLE VI
Acetaminophen Release at 5 minutes from
Enrobed Dose vs Commercial Solid forms

| | Solid form | | | | |
|---|---|---|---|---|---|
| | Enrobed | | | | |
| | Solid form | TYLENOL | | | |
| | Present | RAPID | | Gelatin | Gelatin |
| | Invention | RELEASE | PANADOL | hard | coated |
| | (Example 6) | capsule | ACTIFAST | capsules | tablets |
| APAP | 78 +/− 10 | 82 +/− 10 | 81 +/− 3 | 5 +/− 3 | 0 +/− 0.1 |
| (%) | | | | | |
| released | | | | | |
| in 5 | | | | | |
| minutes | | | | | |

### Example 7

Enrobed solid forms were prepared with acetaminophen in the powder fill material using COMPAP® L, a commercial directly compressible, fast disintegrating powder containing 90% acetaminophen in addition to starch, polyvinylpyrrolidone, stearic acid and CrosPovidone, used as the superdisintegrant, according to its MSDS sheet. Examples 7-1, 7-2, 7-3, 7-4 and 7-5 are examples within the scope of the invention.

For example 7-1: The lower film thickness was about 120 microns, and the upper film thickness was about 90 microns prior to thermoforming. The lower film thermoforming step (140° C., 2 second) produced a solid form of 3 millimeters in height. The upper film thermoforming step (140° C., 2 seconds) gave a film overlap of 2 millimeters. Adhesive 1 was used. The ironing process was by passing the solid form through a hole in a die at 40° C. with a transit time 20 seconds. The enrobed solid form had an average weight of 480 mg (19 mg upper film, 19 mg lower film and 442 mg of fill).

For example 7-2 and 7-5: the lower film thermoforming step (140° C., 1 second) produced a solid form of 2 millimeters in height. The upper film thermoforming step (160° C., 1 second) resulted in a film overlap of 1 millimeter. Adhesive 2 was used. The enrobed solid forms were ironed at 60° C. for 110 seconds. The enrobed solid form 7-2 had an average weight of 314 mg (18.5 mg upper film, 18.5 mg lower film and 277 mg of fill). The enrobed solid form 7-5 had an average weight of 313 mg (18.5 mg upper film, 18.5 mg lower film and 276 mg of fill). 7-2 and 7-5 were taken form the same manufacturing batch. 7-2 were tested immediately after manufacture, 7-5 were tested after 20 months storage at room conditions. The results in table VII show that the release profile of the drug from the solid form of the present invention is stable with time.

For examples 7-3 and 7-4: the lower and upper film thicknesses were about 120 microns prior to thermoforming. The lower film thermoforming step (155° C., 2 seconds) produced a dose cup 3 millimeters in height. The upper film thermoforming step (150° C., 3 seconds) gave a film overlap of 1.5 millimeters. No adhesive was applied on these solid forms. The ironing process was by passing the solid form 7-4 through a hole in a die at 60° C. with a transit time 13.2 seconds. Solid form 7-3 was not passed through the ironing step. The enrobed solid form 7-3 had an average weight of 656 mg (19 mg upper film, 19 mg lower film and 618 mg of fill). The enrobed solid form 7-4 had an average weight of 377 mg (19 mg upper film, 19 mg lower film and 339 mg of fill).

Examples 7-1, 7-2, 7-3 and 7-4 were tested for dissolution at 37° C. according to USP 24 for acetaminophen using 900 ml of phosphate buffer at pH 5.8 in dissolution apparatus 2, paddles. USP specifications for Acetaminophen tablets for immediate release are: not less than 85% of the drug dissolved after 30 minutes (Q). All samples in the following Table VII are examples within the scope of the invention.

TABLE VII
Release of Acetaminophen from Enrobed Solid forms

| Enrobed Solid form | | | | |
|---|---|---|---|---|
| 7-1 | 7-2 | 7-3 | 7-4 | 7-5 |

| Powder fill material | | | | | |
|---|---|---|---|---|---|
| COMPAP ® L | 100% | 100% | 100% | 100% | 100% |
| Lower film thickness | 120 | 120 | 120 | 120 | 120 |
| (microns) | | | | | |
| Upper | 80 | 120 | 120 | 120 | 120 |
| film thickness (microns) | | | | | |
| Solid form weight (milligrams) | 480 | 314 | 656 | 377 | 313 |
| Fill weight | 440 | 277 | 618 | 339 | 276 |
| (milligrams) | | | | | |
| Density (g/ml) of fill | 1.12 | 0.62 | 1.13 | 0.62 | 0.63 |
| APAP (%) in powder fill | 90 | 90 | 90 | 90 | 90 |
| | APAP | APAP | APAP | APAP | APAP |
| | release | released | released | released | released |
| Dissolution time | (%) | (%) | (%) | (%) | (%) |
| 5 minutes | Not tested | 92 ± 5 | 70 +/− 18 | 83 +/− 6 | 89 ± 5 |
| 10 minutes | 71 ± 12 | Not tested | Not tested | Not tested | Not |
| | | | | | tested |
| 15 minutes | Not tested | 99 ± 3 | 89 +/− 5 | 89 +/− 6 | 97 ± 2 |
| Q time (30 min) | 96 +/− 7 | 100 ± 1 | 93 +/− 3 | N/A | 99 ± 1 |

### Example 8

Enrobed solid forms were prepared with powder fill materials using high drug loadings of 98% and 99% of RHODAPAP® acetaminophen powder. The two formulations tested were (1) 99% acetaminophen and 1% colloidal silica and (2) 98% acetaminophen, 1% AC-DI-SOL® and 1% colloidal silica. The thermoforming step (145° C., 2 seconds) formed a dose cup with a 3 millimeter height. The upper film thermoforming step (160° C., 4 seconds) gave a film overlap of 1 millimeter. Adhesive 1 was used. The ironing process used a 20 second dwell (40° C.). Example 8-1 is a comparative example, and Example 8-2 is an example within the scope of the invention.

Examples 8-1 and 8-2 were tested for dissolution at 37° C. according to USP 24 for acetaminophen using 900 ml of phosphate buffer at pH 5.8 in dissolution apparatus 2, paddles. USP specifications for Acetaminophen tablets for immediate release are: not less than 85% of the drug dissolved after 30 minutes (Q). 8-1 in the Table below is a comparative example, and 8-2 in the Table below is an example of the present invention.

TABLE VIII
Enrobed Solid forms containing Acetaminophen

| | Enrobed dose | |
|---|---|---|
| | 8-1 | 8-2 |
| Powder Fill formulation | | |
| RHODAPAP ® powder (%) | 99 | 98 |
| AC-DI-SOL ® (%) | 0 | 1 |
| CAB-O-SIL ® M-P5 (%) | 1 | 1 |
| Lower film thickness (microns) | 120 | 123 |
| Upper film thickness (microns) | 80 | 83 |
| Dosage weight (milligrams) | 501 | 520 |
| Fill weight (milligrams) | 455 | 472 |
| Density (g/ml) of fill | 0.84 | 0.86 |
| Dissolution time | % APAP released | % APAP released |
| 5 minutes | Not tested | 79 +/− 15 |
| 8 Minutes | Not Tested | 92 +/− 6 |
| 10 minutes | 69 +/− 11* | Not Tested |

*Does not meet USP specifications at 30 minutes for Acetaminophen Immediate Release tablets.

### Example 9

Enrobed solid forms were prepared with acetaminophen in the powder fill material using COMPAP® L, a commercial directly compressible, fast disintegrating powder containing 90% acetaminophen in addition to starch, polyvinylpyrrolidone, stearic acid and CrosPovidone, used as the superdisintegrant, according to its MSDS sheet. Example 9-1, 9-2, 9-3 are examples within the scope of the invention.

The lower and upper film thicknesses were about 120 microns prior to thermoforming. The lower film thermoforming step (155° C., 2 seconds) produced a dose cup 2.7 millimeters in height. The upper film thermoforming step (165° C., 3 seconds) gave a film overlap of 1.5 millimeters for example 9-3, and 1.3 mm for examples 9-1 and 9-2. No adhesive was applied on these solid forms. The ironing process was by passing the solid form through a hole in a die at 60° C. with a transit time 79.2 seconds. The enrobed solid form 9-1 had an average weight of 553 mg (19 mg upper film, 19 mg lower film and 515 mg of fill). The enrobed solid form 9-2 had an average weight of 494 mg (19 mg upper film, 19 mg lower film and 456 mg of fill). The enrobed solid form 9-3 had an average weight of 403 mg (19 mg upper film, 19 mg lower film and 365 mg of fill). All samples in the Table below are examples within the scope of the invention.

TABLE IX
Release of Acetaminophen from Enrobed Solid forms

| | Enrobed Solid form | | |
|---|---|---|---|
| | 9-1 | 9-2 | 9-3 |
| Powder fill material | | | |
| COMPAP ® L | 100% | 100% | 100% |
| Lower film thickness | 120 | 120 | 120 |
| (microns) | | | |
| Upper film thickness | 120 | 120 | 120 |
| (microns) | | | |
| Solid form weight | 553 | 494 | 403 |
| (milligrams) | | | |
| Fill weight (milligrams) | 515 | 456 | 365 |
| Density (g/ml) of fill | 1.00 | 0.90 | 0.71 |
| APAP (%) in powder fill | 90 | 90 | 90 |
| APAP release (%) after 5 | 87 +/− 11 | 77 +/− 15 | 76 +/− 13 |
| minutes | | | |

### Example 10

Dissolution of compacted powder fill compositions containing ibuprofen with and without the superdisintegrant AC-DI-SOL® croscarmellose sodium and with and without the wetting agent Docusate sodium (DS) or Sodium Lauryl Sulfate (SLS) was measured in phosphate buffer at pH 7.2 as specified in USP 24 for ibuprofen tablets for Immediate Release. The powder fill compositions were: (1) 76% ibuprofen, 22.6% AVICEL® PH101 microcrystalline cellulose, 0.4% Docusate sodium and 1% talc in a granulate form, (2) 76% ibuprofen, 20.6% AVICEL® PH101 microcrystalline cellulose, 0.4% Docusate sodium, 2% Ac-Di-Sol and 1% talc in a granulate form, (3) 76% ibuprofen, 23% AVICEL® PH200 microcrystalline cellulose, and 1% talc in a powder form (formulation from example 2 (1), (4) 50% ibuprofen, 48% AVICEL® PH102, 1% Sodium lauryl sulfate and 1% talc, (5) 50% ibuprofen, 46% AVICEL® PH102, 2% AC-DI-SOL®, 1% Sodium lauryl sulfate and 1% talc. The powder compacts were prepared using 250 mg of powder fill.

Example 10-4 is a comparative example, and examples 10-1, 10-2, 10-3, 10-5 and 10-6 are examples within the scope of the invention.

Table X shows the % ibuprofen released during dissolution at 5 minutes at 37° C. according to USP 24 for Ibuprofen immediate release tablets using 900 ml of phosphate buffer at pH 7.2 in dissolution apparatus 2, paddles as a function of density for samples with and without the AC-DI-SOL® croscarmellose sodium and with or without the wetting agent. 10-4 is a comparative example and all other samples in the Table below are examples of the present invention.

TABLE X
Effect of the addition of a wetting agent in the formulation on ibuprofen release

| | Compaction | | | | Presence | Ibuprofen | Ibuprofen |
|---|---|---|---|---|---|---|---|
| | Pressure | Density | Formulation | Wetting | of | (%) | (%) at |
| Eg | (MPa) | (g/ml) | number | agent | Ac-Di-Sol | at 5 min | Q (60 min) |
| 10-1 | 7 | 0.83 | 1 | DS | no | 84 +/− 6 | 98 +/− 4 |
| 10-2 | 22 | 1.08 | 2 | DS | yes | 69 +/− 8 | 91 +/− 2 |
| 10-3 | 5 | 0.86 | 2 | DS | yes | 82 +/− 10 | 92 +/− 3 |
| 10-4 | 10 | 0.84 | 3 | No | No | 60 +/− 21 | 83 ± 2 |
| 10-5 | 6 | 0.68 | 4 | SLS | No | 90 +/− 11 | N/A |
| 10-6 | 6 | 0.69 | 5 | SLS | yes | 96 +/− 4 | N/A |

### Example 11

Enrobed solid forms were prepared with the following blends: (1) 95% Aspirin, 3% talc and 2% Ac-Di-Sol (2) 98% Ketoconazole and 2% Ac-Di-Sol (3) 98% Theophylline and 2% Ac-Di-Sol. Example 11-1, 11-2, 11-3 and 11-4 are examples within the scope of the invention.

The lower and upper film thicknesses were about 120 microns prior to thermoforming.

In examples 11-1, the lower film thermoforming step (155° C., 2 seconds) produced a dose cup 3.1 millimeters in height. The upper film thermoforming step (165° C., 4.5 seconds) gave a film overlap of 1.5 millimeters. No adhesive was applied. In examples 11-2 and 11-3, the lower film thermoforming step (155° C., 3 seconds) produced a dose cup 2.5 millimeters in height. The upper film thermoforming step (165° C., 3 seconds) gave a film overlap of 1.5 millimeters. Adhesive 1 was used. In examples 11-4, the lower film thermoforming step (165° C., 2 seconds) produced a dose cup 2.5 millimeters in height. The upper film thermoforming step (165° C., 3 seconds) gave a film overlap of 1.5 millimeters. Adhesive 1 was used.

In example 11-1 the ironing process was by passing the solid form through a hole in a die at 60° C. with a transit time of 14.5 seconds. In examples 11-2, 11-3 and 11-4 the ironing process was by passing the solid form through a hole in a die at 40° C. with a transit time of 20 seconds.

The enrobed solid form 11-1 had an average weight of 783 mg (19 mg upper film, 19 mg lower film and 745 mg of fill). The enrobed solid form 11-2 had an average weight of 580 mg (19.5 mg upper film, 19.5 mg lower film and 541 mg of fill). The enrobed solid form 11-3 had an average weight of 540 mg (19.5 mg upper film, 19.5 mg lower film and 501 mg of fill). The enrobed solid form 11-4 had an average weight of 421 mg (19.5 mg upper film, 19.5 mg lower film and 382 mg of fill).

Aspirin solid forms were tested for dissolution at 37° C. using 900 ml of acetate buffer at pH 4.5 in dissolution apparatus 2, paddles. USP specifications for Aspirin tablets for immediate release are: not less than 85% of the drug dissolved after 30 minutes (Q). Ketoconazole solid forms were tested for at 37° C. according to USP 28 for Ketoconazole immediate release tablets using 900 ml of 0.1N hydrochloric acid in dissolution apparatus 2, paddles. USP specifications for Ketoconazole immediate release tablets are: not less than 85% of the drug dissolved after 30 minutes (Q). Theophylline solid forms were tested for at 37° C. according to USP 28 for Theophylline immediate release tablets using 900 ml of water in dissolution apparatus 2, paddles. USP specifications for Theophylline immediate release tablets are: not less than 85% of the drug dissolved after 45 minutes (Q). The samples in the Table below are all examples within the scope of the invention.

TABLE XI
Release of Drugs from high drug loaded Enrobed Solid forms

| | Enrobed Solid form | | | |
|---|---|---|---|---|
| | 11-1 | 11-2 | 11-3 | 11-4 |
| Formulation | (1) | (2) | (3) | (3) |
| Drug | Aspirin | Ketoconazole | Theophylline | Theophylline |
| Drug loading | 95 | 98 | 98 | 98 |
| (%) | | | | |
| Lower film | 120 | 120 | 120 | 120 |
| thickness | | | | |
| (microns) | | | | |
| Upper film | 120 | 120 | 120 | 120 |
| thickness | | | | |
| (microns) | | | | |
| Solid form | 783 | 580 | 540 | 421 |
| weight | | | | |
| (milligrams) | | | | |
| Fill weight | 745 | 541 | 501 | 382 |
| (milligrams) | | | | |
| Density (g/ml) | 1.34 | 1.06 | 1.04 | 0.77 |
| of fill | | | | |
| Drug release | 44 +/− 7 | 60 +/− 15 | 80 +/− 23 | 61 +/− 26 |
| (%) after 5 | | | | |
| minutes | | | | |
| Drug release | 93 +/− 8 | 99 +/− 2 | 100 +/− 1 | 93 +/− 2 |
| (%) at Q time | | | | |

The invention has been illustrated by detailed description and examples of the preferred embodiments. Various changes in form and detail will be within the skill of persons skilled in the art. Therefore, the invention must be measured by the claims and not by the description of the examples or the preferred embodiments.

1. A solid form comprising at least one film enrobing a compacted fill material wherein:
i) said compacted fill material comprises at least one active material and at least one of a disintegrant and a wetting agent;
ii) said solid form has a weight loss that is less than 1% during a 30 minute USP Friability Test USP 29 Test Number 1216; and
iii) said compacted fill material in said solid form has a density of at least 0.5 g/ml based on the total solid volume of the solid form and a tensile strength of less than 0.9 MPa; and
iv) the at least one active material within the solid form exhibits immediate release.

2. A solid form comprising at least one film enrobing a compacted fill material wherein the compacted fill material comprises at least one active material and at least one of a disintegrant and a wetting agent and the compacted fill material is selected from a pharmaceutical product, food product, a veterinary product, a cosmetic, an appetite suppressant, a detergent product and a nutraceutical product, the said solid form shows a weight loss that is less than 1% during a 30 minutes United States Pharmacopeia Friability Test USP 29 Test Number 1216 (page 3046) and the said solid form dissolves with an immediate release profile.

3. A solid form according to claim 1 or claim 2 wherein the active material in said solid form comprises at least one pharmaceutical active and said at least one pharmaceutical active has a mean dissolution which meets the USP dissolution specifications in the test specified in the USP Edition 29 Test Number 711 at page 2673 for said active material when said active material is placed in a dissolution medium as specified in the USP dissolution specification or selected from dissolution media specified in the USP according to the solubility properties of said active material.

4. A solid form according to claim 1 or claim 2 wherein said compacted fill material comprises a moderate to high amount of said at least one active material.

5. A solid form according to claim 4 wherein said compacted fill material comprises a high amount of said at least one active material.

6. A solid form according to claim 1 or claim 2 wherein the disintegrant comprises at least one super disintegrant and said fill material has a density of 0.5 to 1.1 g/ml, said at least one active material has a solubility of 1 g of active material in ≧10 g of water, and wherein at least one of said active materials has a mean dissolution of at least 75% in 300 seconds when the active material is tested in the test specified in the USP Edition 29 Test Number 711 at page 2673 for said active material in a dissolution medium as specified in the USP dissolution specification or, if the medium is not specified in the said specification, the dissolution medium is selected according to the solubility properties of the active material from dissolution media specified in the USP or from: (i) water, (ii) 0.1 M HCI or (iii) phosphate buffer having a pH between 5.8 and 8.0.

7. A solid form according to claim 6, wherein said super disintegrant comprises at least one of croscarmellose sodium, sodium starch glycolate, crosslinked polyvinyl pyrrolidone or low substituted hydroxypropyl cellulose.

8. A solid form according to claim 6 wherein said at least one active material has a mean dissolution of at least 85% in 300 seconds in any of said dissolution media.

9. A solid form according to claim 6 wherein said at least one active material has a mean dissolution of at least 95% in 300 seconds in any of said dissolution media.

10. A solid form according to claim 6, wherein said at least one active material has a solubility in water of 1 g active in 10 to 30 g water.

11. A solid form according to claim 6, wherein said at least one active material has a solubility in water selected from the range of 1 g active in 30 to 100 g water.

12. A solid form according to claim 6 wherein said at least one active material has a solubility in water of 1 g active in 100 to 1,000 g water.

13. A solid form according to claim 6, wherein said at least one active material has a solubility of 1 g active in 1,000 to 10,000 g water.

14. A solid form according to claim 6, wherein said at least one active material has a solubility in water of 1 g active in more than 10,000 g water.

15. A solid form according to claim 6 wherein said super disintegrant is present in an amount of 0.1 to 10% by weight of the compacted fill material.

16. A solid form according to claim 1 or claim 2 wherein the compacted fill material in said solid form has a density from 0.55 to 1.04 g/ml.

17. A solid form according claim 1 or claim 2 wherein the compacted fill material has a density from 0.75 to 1 g/ml.

18. A solid form according claim 1 or claim 2 wherein the solid form has a tensile strength of at least 1.3 MPa.

19. A solid form according claim 1 or claim 2 wherein said at least one active material is present in an amount of at least 30% by weight of the compacted fill material.

20. A solid form according to claim 1 or claim 2 further comprising a filler.

21. A solid form according to claim 20, wherein said filler comprises at least one of microcrystalline cellulose, dicalcium phosphate, lactose calcium carbonate, calcium phosphate dibasic anhydrous, calcium phosphate dibasic dehydrate, calcium phosphate tribasic, powdered cellulose, silicified microcrystalline cellulose, cellulose acetate, compressible sugar, confectioners sugar, dextrin, dextrose, ethylcellulose, fructose, lactitol, starch, pregelatinized starch, sucrose, talc, xylitol, maltodextrin, magnesium carbonate, maltose, mannitol, polydextrose, sodium alginate, sodium chloride, sorbitol, sucrose, sugar spheres, acacia, carrageenan, carbomer, chitosan, hydroxypropylmethylcellulose, carboxymethylcellulose sodium, gelatin, guar gum, hydroxyethyl cellulose, hydroxypropyl cellulose, methylcellulose, povidone, zein, citric acid, sodium bicarbonate, alginic acid, carboxymethylcellulose calcium, colloidal silicon dioxide, low substituted hydroxypropyl cellulose.

22. A solid form according to claim 20, wherein said filler is present in an amount less than 70% by weight of the compacted fill material.

23. A solid form according to claim 22, wherein said filler is present in an amount of 1 to 10% by weight of the compacted fill material.

24. A solid form according to claim 1 or claim 2, wherein said solid form does not contain a filler.

25. A solid form according to claim 1 or claim 2, wherein said active material comprises at least one of an analgesic, antiangina, antianaemia, antibiotic, antiarrhythmic, antidiarrheal, antidiuretic, antidepressant, antiemetic, antifungal, antirheumatic, antiviral, antiprotozoal, antihistamine, antihypertensive, anti-inflammatory, antimigraine, antinausea, antispasmodic, anxiolytic, beta blocker, calcium channel blocker, sedative, hypnotic, antipsychotic, bronchodilator, decongestant, cough expectorant, cough suppressant, antiasthma drug, corticosteroid, actives for treatment of cough or common cold, muscle relaxant, erectile dysfunction active or motion sickness active.

26. A solid form according to claim 1 or claim 2, comprising at least two active materials wherein the active materials are selected from:
i) an antibiotic in combination with a decongestant, an anti-inflammatory, a cough expectorant, a cough suppressant or an active for treatment of cough or common cold, a proton pump inhibitor;
ii) an anti-HIV, anti-malaria active material, an anti-hypertension and anti-cholesterol,
iii) two or more active materials from the same class of active materials, the class being selected from respiratory actives, gastronintestinal actives, cardiovascular actives, antidiabetes actives, central nervous system actives, anti-infection actives, anti-viral actives, analgesics, anti-inflammatory actives, antibiotics, cough suppressants, expectorants, mucolytics, and nasal decongestants.

27. A solid form according to claim 1 or claim 2, wherein the said at least one active material comprises at least one of paracetamol, pseudoephedrine, acravastine, lamivudine, abacavir, pravastatin, Roziglitazone, ezetimibe, Clavulanate, sulfamethoxazole, benazepril, Valsartan, Irbesartan, Losartan, Dutasteride, tamsolusin, Atazanavir, ritonavir, propoxyphene, Hydrocodone, Metocarbamol, Memantine, Donepezil, Glyburide, Pioglytazone, Glimepiride, Benazepril, Torcetrapib, Eprosartan, Telmisartan, Olmesartan, Lopinavir, Emtricitabine, Tenofovir, Amprenavir, Tipranavir, Atovaquone, Proguanil, 5-aminosalicylic acid, 4-aminophthalic acid, Bismuth citrate, Bismuth subsalicylate, Montelukast, pseudoephedrine, Guaifenesin, ibuprofen, nifedipine, betamethasone acetate, methylprednisolone, dextromethorphan, cinnarazine, simvastatin, ciprofloxacin, glipizide, risperidone, glibenclamide, fenofibrate, isosorbide mononitrate, isosorbide dinitrate, acetazolamide, levothyroxine sodium, omeprazole, aspirin, codeine, dihydroergotamine, diazepam, theophylline, sildenafil citrate, vardenafil hydrochloride, amlodipine besylate, zolpidem tartrate, acetaminophen, methocarbamol, ramipril, digoxin, enalapril maleate, fluoxetine hydrochloride, fexofenadine hydrochloride, olanzapine, methyldopa, hydrochlorothiazide, timolol maleate, alendronate sodium, thiabendazole, rofexocib, dicoflenac, bepridil hydrochloride, atorvastatin hydrochloride, sertraline hydrochloride, famciclovir monohydrate, nabumetone, cimetidine, ketoprofen, etodolac, amiodarone hydrochloride, indomethacin, cefaclor, diltiazem, verapamil, felodipine, isradipine, nicardipine, prazosin, disopyramide, pentoxifilline, venlafaxine, alfuzosin, doxazosin, famotidine, ranitidine, pirenzipine, lansoprazole, loperamide, sulfasalazine, prednisolone, furosemide, amiloride, triamterene, verapamil, atenolol, propranolol, captopril, glyceryl trinitrate, caffeine, aminophylline, cetirizine, loratadine, chlorpheniramine maleate, diphenhydramine, dothiepin, amitriptyline, pheneizine, paroxetine, fenfluramine, dimenhydrinate, ondansetron, domperidone, metoclopramide, tramadol, dihydrocodeine, pethidine, sumatriptan, amoxicillin, ampicillin, cefuroxime, cephalexin, tetracycline, erythromycin, co-trimoxazole, sulphadiazine, trimethoprim, nitrofurantoin, fluconazole, ketoconazole, acyclovir, zidovudine, chloroquine, mefloquin, metronidazole, metformin, chlorpropamide, ferrous sulphate, azapropazone, fenbufen, flurbiprofen, ketoprofen, naproxen, piroxicam, mefanamic acid, celecoxib, licofelone, tadalafil, mycophenolate, valgancyclovir, valacyclovir, sevelamer, metaxolone, nelfinavir, duranavir, tipranavir, levetiracetam, capecitabine, moxifloxacin, morphine, levofloxacin, clarithromycin, pregabalin, esomeprazole, quetiapine, efavirenz, oxcarbazepine, colesevelam, zileuton, nitazoxanide, clofibrate, praziquantel, sucralfate, cefprozil, indinavir, ganciclovir, oxaprozin, divalproex, cefadroxil, felbamate, potassium chloride, saquinavir, fosamprenavir, hydroxyurea, gabapentin, niacin, omega-3 acid ethyl esters, calcium acetate, progesterone, procainamide, delavirdine, ribavirin, propafenone, eprosartan, tocainide, tinidazole, choline magnesium trisalicylate, azithromycin, linezolid, lorazepam, oxazepam, lormetazepam, flunitrazepam, haloperidol, triptorelin, leuprorelin, lanreotide acetate, octreotide acetate, methylxanthin, tamsulosin, codeine hydrochloride, dextromoramide tartrate, ethymorphine hydrochloride, magnesium salicylate, methadone hydrochloride, oxycodone hydrochloride, sufentanil citrate, ephedrine, tramazoline hydrochloride, brompheniramine maleate, emedastine fumarate, and pharmaceuticaly or nutraceuticaly acceptable salts, acids, esters, isomers, and metabolites thereof.

28. A solid form according to claim 1 or claim 2, comprising at least two active materials wherein the active materials are selected from: paracetamol and caffeine; aspirin and paracetamol; paracetamol and pseudoephedrine; paracetamol and phenylephrine; ibuprofen and codeine; ibuprofen and pseudoephedrine; paracetamol and diphenhydramine; acravistine and pseudoephedrine; paracetamol and dextromethorphan; paracetamol and guaphenesin; paracetamol, caffeine, aspirin; aspirin and caffeine; zidovudine, iamivudine and abacavir; pravastatin and aspirin; lamivudine and zidovudine; roziglitazone and metformin; ezetimibe and fenofibrate; amoxicillin and clavulanate; trimetoprim and sulfamethoxazole; amlodipine and benazepril; valsartan and hydrochlorothiazide; irbesartan and hydrochlorothiazide; losartan and hydrochlorothiazide; fenofibrate and metformin; abacavir and lamivudine; dutasteride and tamsolusin; atazanavir and ritonavir; ritonavir and saquinavir; propoxyphene and paracetamol; hydrocodone and paracetamol; tramadol and paracetamol; metocarbamol and paracetamol; memantine and donepezil; glyburide and metformin; pioglytazone and metformin; rosiglitazone and glimepiride, benazepril and hydrochlorothiazide; atorvastatin and torcetrapib; eprosartan and hydrochlorothiazide; amlodipine and atorvastatin; ezetimibe and simvastatin; telmisartan and hydrochlorothiazide; olmesartan and hydrochlorothiazide; lopinavir and ritonavir; emtricitabine and tenofovir; fosamprenavir and ritonavir; amprenavir and ritonavir; tipranavir and ritonavir; atovaquone and proguanil; lansoprazole, amoxicillin and clarithromycin; lansoprazole and naproxen; 5-aminosalicylic acid and 4-aminophthalic acid acid; clarithromycin, ranitidine and bismuth citrate; bismuth subsalicylate, metronidazole and tetracycline; montelukast and loratadine; fexofenadine and pseudoephedrine; Guaifenesin and pseudoephedrine.

29. A solid form according to claim 1 or claim 2 comprising a wetting agent selected from at least one of hypromellose, docusate sodium, sodium lauryl sulfate, polyoxyethylene sorbitan fatty acid esters, sorbitan esters, polyoxyethylene alkyl ethers, dioctyl calcium sulfosuccinate, povidone, cyclodextrins, poloxamers, glyceryl monostearate,

30. A solid form according to claim 1 or claim 2 comprising a wetting agent which is present in an amount of 0.01 to 10.0 wt % based on the total weight of the compacted fill material.

31. A solid form according to claim 1 or claim 2 in which the compacted fill material comprises a wetting agent and either a disintegrant or a super-disintegrant.

32. A solid form according to claim 1 or claim 2 wherein the compacted fill material has a tensile strength less than 0.2 MPa.

33. A solid form according to claim 1 or claim 2, wherein said disintegrant is present in an amount of 0.1 to 25% by weight of the compacted fill material.

34. A solid form according to claim 1 or claim 2 wherein said at least one active material has a solubility in water of 1 g active in 1-10 g water.

35. A solid form according to claim 1 or claim 2 wherein said at least one active material has a solubility in water of 1 g active in less than 1 g water.

36. A solid form according to claim 1 or claim 2, wherein said active material is a powder material comprising at least one of granules, micronized powders, spray-dried powders and freeze-dried powders and pellets.

37. A solid form according to claim 1 or claim 2, wherein said disintegrant comprises at least one of croscarmellose sodium, sodium starch glycolate, crosslinked polyvinyl pyrrolidone or low substituted hydroxypropyl cellulose, alginic acid, calcium phosphate, carboxymethylcelluloses, powdered cellulose, chitosan, colloidal silicon dioxide, guar gum, magnesium aluminium silicate, methylcellulose, microcrystalline cellulose, povidone, sodium alginate, starch, pregelatinised starch.

38. A solid form according to any claim 1 or claim 2, wherein the film enrobing the compacted fill material is a water-soluble film.

39. A solid form according to claim 1 or claim 2 for use in a method of treatment of the human or animal body by therapy.

40. A method of treatment of the human or animal body comprising administering a solid form according to claim 1 or claim 2 to the human or animal body.
